(19) United States
(12) Patent Application Publication
(10) Pub. No.: US 2026/0364115 A1
(43) Pub. Date: Nov. 19, 2026

(54) ANTI-INFLAMMATORY COMPOSITION COMPRISING AS ACTIVE INGREDIENT EXTRACELLULAR VESICLES ISOLATED FROM TONSIL-DERIVED MESENCHYMAL STEM CELLS STIMULATED WITH PDRN

(71) Applicant: HYUNDAEMEDITECH CO., LTD., Wonju-si, Gangwon-do (KR)

(72) Inventor: Mi Hee SONG, Wonju-si, Gangwon-do (KR)

(21) Appl. No.: 19/477,461

(22) PCT Filed: Jan. 30, 2024
(86) PCT No.: PCT/KR2024/001435

§ 371 Date: Nov. 11, 2025

(30) Foreign Application Priority Data
Apr. 21, 2023 (KR) 10-2023-0052531

(51) Int. Cl. A61K 35/28; C12N 5/0775

(52) U.S. Cl. CPC A61K 35/28; C12N 5/0668; C12N 2500/40

(57) ABSTRACT

The present invention relates to an anti-inflammatory composition comprising, as an active ingredient, extracellular vesicles isolated from tonsil-derived mesenchymal stem cells stimulated with PDRN. Particularly, an anti-inflammatory active composition comprising extracellular vesicles isolated from tonsil-derived mesenchymal stem cells, according to the present invention, has excellent inflammatory cytokine TNF-α inhibitory activity and excellent anti-inflammatory cytokine IL-10 increasing activity, and thus can be effectively used to prevent, inhibit, alleviate, ameliorate or treat inflammation.

### TECHNICAL FIELD

The present invention relates to an anti-inflammatory composition including as an active ingredient extracellular vesicles (EVs) isolated from tonsil-derived mesenchymal stem cells.

### BACKGROUND ART

Extracellular vesicles (EVs) are nano-sized vesicles secreted by all cells into the external environment for intercellular information exchange. EVs contain a variety of biologically active substances, including proteins, lipids, nucleic acids, and metabolites. Recently, substances included in EVs have been identified through high-throughput analysis. In addition, EVs reflect the state of the cells which they originate from and have been found in various bodily fluids. EVs interact with target cells to perform physiological functions under normal conditions, while others perform pathological functions that cause diseases. Recently, research on the diagnosis and treatment of diseases using EVs has been actively conducted.

Meanwhile, inflammation is a defense response of the body that occurs in response to pathological conditions caused by physical or chemical trauma, infection by bacteria, fungi, or viruses, and various allergens. The inflammatory response occurs as part of the innate immune response. Various substances and physiological and chemical phenomena are involved in the inflammatory response, and recent studies have shown that various inflammatory cytokines play an important role in the inflammatory response. Major cytokines involved in the inflammatory response include IL-1β, TNF-α, IL-6, IL-8, IL-12, and IFN-β, and the increase in their expression and secretion amount and activation are related to a series of complex physiological responses such as secretion of inflammatory mediators, immune cell infiltration, cell migration, and tissue destruction, as well as symptoms such as erythema, edema, fever, and pain.

Generally, the inflammatory response is not a major problem and the diseased area is recovered to normal when the infectious agent is removed and the damaged tissue is regenerated. However, when the infectious agent is not removed or when the inflammatory response is excessive or persistent due to internal substances, it may cause acute or chronic inflammatory diseases.

Nonsteroidal anti-inflammatory drugs, steroidal anti-inflammatory drugs, neuropeptide antagonists, cyclooxygenase (COX) inhibitors, antihistamines, and immunosuppressants such as cyclosporin A are used to alleviate or treat inflammatory responses or associated inflammatory diseases. However, these drugs may cause adverse effects such as skin atrophy, vasodilation, depigmentation, hypersensitivity reactions, drug resistance, and neutropenia.

Therefore, there is a growing need to develop agents with superior anti-inflammatory effects that overcome the limitations of existing therapeutic agents.

### DETAILED DESCRIPTION OF THE INVENTION

### Technical Problem

One object of the present invention is to provide an anti-inflammatory composition including extracellular vesicles (EVs) isolated from tonsil-derived mesenchymal stem cells to address the above-described problems.

Another object of the present invention is to provide a pharmaceutical composition for preventing or treating an inflammatory disease, including the anti-inflammatory composition.

Still another object of the present invention is to provide an anti-inflammatory functional cosmetic composition including the anti-inflammatory composition.

Yet another object of the present invention is to provide a health functional food composition for preventing or ameliorating of an inflammatory disease, including the anti-inflammatory composition

Yet another object of the present invention is to provide a quasi-drug composition for preventing or ameliorating of an inflammatory disease, including the anti-inflammatory composition.

Mesenchymal stem cells are known to play a crucial role in maintaining homeostasis and regeneration in all tissues where blood vessels are distributed. Their mechanisms of action include vascular protection, angiogenesis, tissue cell protection, tissue stem cell mobilization, and inflammation suppression, in which mesenchymal stem cells play an important mediating role. It is already known that a significant portion of the tissue regeneration function of these mesenchymal stem cells is mediated by growth factors and particles collectively referred to as EVs, secreted from these cells. Polydeoxyribonucleotide (PDRN) is a degraded form of DNA that is not normally observed outside of cells. However, PDRN is sometimes released from cells destroyed by excessive inflammation or from infectious agents containing DNA such as viruses or bacteria, and in these situations, it serves as a signaling substance for tissue regeneration in tissue cells. Due to these characteristics, PDRN is used as a means to induce tissue regeneration by mimicking an inflammatory environment in various diseases. The present invention aims to improve the function of mesenchymal stem cells related to tissue regeneration, particularly, inflammation control function, by treating mesenchymal stem cells with PDRN to add a stimulation signal of tissue damage to the mesenchymal stem cells, and to isolate EVs contained in the culture medium of mesenchymal stem cells and utilize them as an active ingredient for treatment.

### Technical Solution

To address the above-described problems, the present invention provides an anti-inflammatory composition including extracellular vesicles (EVs) isolated from tonsil-derived mesenchymal stem cells.

The present invention also provides a pharmaceutical composition for preventing or treating an inflammatory disease, including the anti-inflammatory composition

The present invention also provides an anti-inflammatory functional cosmetic composition including the anti-inflammatory composition.

The present invention also provides a health functional food composition for preventing or ameliorating an inflammatory disease, including the anti-inflammatory composition.

The present invention also provides a quasi-drug composition for preventing or ameliorating inflammatory diseases, including the anti-inflammatory composition.

Hereinafter, the present invention will be described in more detail through examples. However, these examples are provided merely to illustrate the present invention, and therefore, the scope of the present invention should not be construed as being limited by these examples.

### Advantageous Effects

The anti-inflammatory composition including extracellular vesicles (EVs) isolated from tonsil-derived mesenchymal stem cells of the present invention exhibits excellent anti-inflammatory effects.

Therefore, the anti-inflammatory composition including as an active ingredient EVs isolated from tonsil-derived mesenchymal stem cells of the present invention can be used to effectively prevent, suppress, alleviate, ameliorate, or treat inflammatory responses or an inflammatory disease.

Furthermore, by stimulating tonsil-derived mesenchymal stem cells with polydeoxyribonucleotide (PDRN) during the culture of the stem cells, the anti-inflammatory efficacy of the EVs subsequently purified or isolated can be enhanced. However, the present invention is not limited to tonsil-derived mesenchymal stem cells and can be extended to mesenchymal stem cells derived from various tissues.

Meanwhile, the scope of the present invention is not limited by the above-described effects. The effects of the present invention are not limited to the above-described examples, and a wider range of effects are included within the present specification.

### DESCRIPTION OF THE DRAWINGS

FIGS. 1 and 2 are diagrams illustrating the results of physical property analysis of stem cell-derived extracellular vesicles (EVs) according to one embodiment of the present invention, showing the particle size distribution and particle count obtained by a nanoparticle tracking analysis (NTA).

FIG. 3 is a diagram illustrating the TNF-α production inhibiting effect of the anti-inflammatory composition including EVs isolated from tonsil-derived mesenchymal stem cells according to one embodiment of the present invention.

FIG. 4 is a diagram illustrating the influence of residual IL-10 amount in the culture medium itself on the results according to one embodiment of the present invention.

FIG. 5 is a diagram illustrating the IL-10 production increasing effect of the anti-inflammatory composition including EVs isolated from tonsil-derived mesenchymal stem cells according to one embodiment of the present invention.

### MODES OF THE INVENTION

The advantages and features of the present invention and methods for achieving them will become apparent with reference to the embodiments described in detail below together with the accompanying drawings. However, the present invention is not limited to the embodiments disclosed below, but may be implemented in various different forms, and the present embodiments are provided only to make the disclosure of the present invention complete and to fully inform those skilled in the art of the scope of the invention, and the present invention is defined only by the scope of the claims.

The present invention is described in more detail below in the following embodiments. However, the following embodiments are intended to illustrate the content of the present invention and do not limit or restrict the scope of the present invention. It is construed that what can be readily inferred by those skilled in the art from the detailed description and embodiments of the present invention falls within the scope of the present invention. The references cited herein are hereby incorporated by reference into the present invention.

Throughout this specification, when a part is said to “comprise” a certain component, unless specified otherwise, this means that it may further include other components, rather than exclude other components.

The present invention provides an anti-inflammatory active composition including extracellular vesicles (EVs).

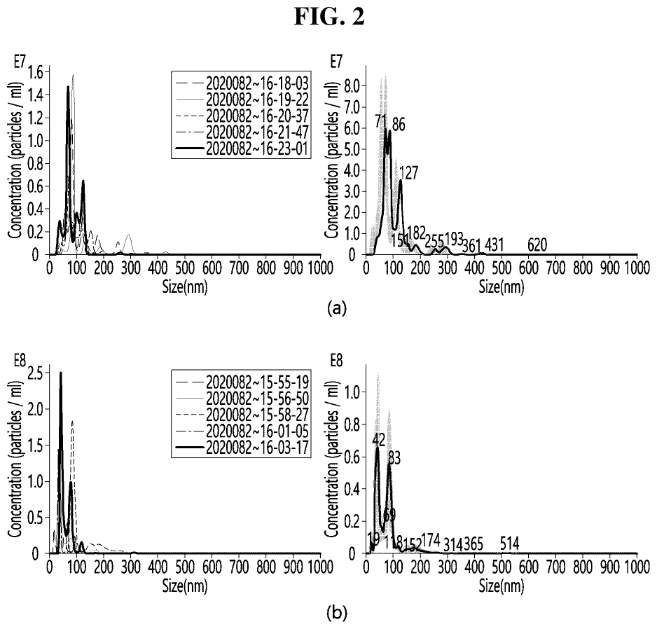

As confirmed in Example 4 and FIGS. 2 and 3, the anti-inflammatory active composition including EVs of the present invention exhibits significant anti-inflammatory activity, demonstrating excellent inhibitory activity against inflammatory cytokine TNF-α and excellent enhancing activity on anti-inflammatory cytokine IL-10, as both are key factors involved in inflammatory responses.

As used herein, the term “EV” is used to incorporate membrane-derived vesicles, ectosomes, shedding vesicles, microparticles, microvesicles, exosomes, secretomes, or equivalents thereof.

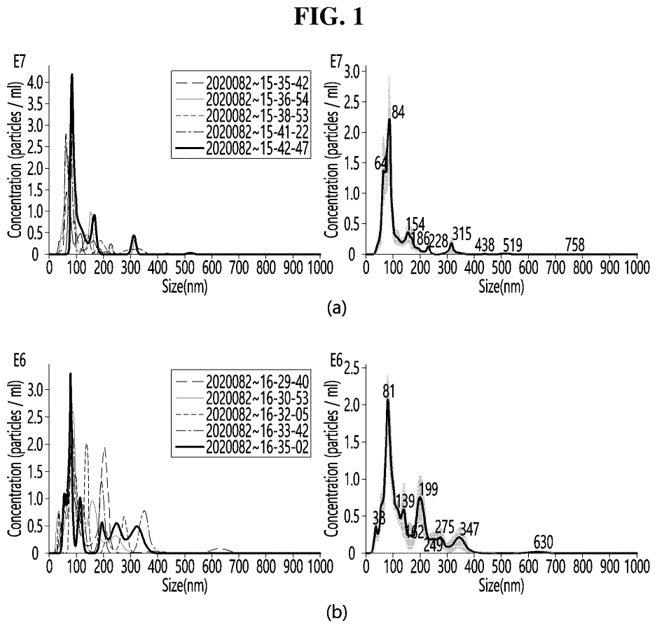

As shown in Example 3 and FIGS. 1 and 2, the particle size of the EV fraction of the present invention may have an average diameter of 10 to 400 nm, more preferably 50 to 300 nm, but is not limited thereto.

As used herein, the term “exosome” refers to vesicles having a size of tens to hundreds of nanometers (preferably approximately 30 to 200 nm) made of a phospholipid bilayer with the same structure as the cell membrane (however, the particle size of the exosome may vary depending on the type of stem cell to be isolated, the isolation method, and the measurement method) (Vasiliy S. Chernyshev et al., “Size and shape characterization of hydrated and desiccated exosomes”, Anal Bioanal Chem, (2015) DOI 10.1007/s00216-015-8535-3), and exosomes contain proteins, mRNA, miRNA, DNA, and the like, which are called exosome cargo. Exosome cargo includes a wide range of signaling factors, and these signaling factors are known to be cell type-specific and differently regulated depending on the environment of the secreting cell. Exosomes are intercellular signaling mediators secreted by cells, and various cell signals transmitted therethrough are known to regulate cell behavior, including activation, growth, migration, differentiation, dedifferentiation, apoptosis, and necrosis of target cells. The exosomes used in the present invention may be isolated from stem cell cultures using various methods known in the art, but are not limited thereto, and of course, those obtained by various separation methods as described above may be used in the anti-inflammatory composition of the present invention.

As used herein, the term “inflammation” refers to the pathological condition of an abscess formed by the invasion of an external infectious agent (bacteria, fungi, viruses, various types of allergens).

Furthermore, the present invention provides a pharmaceutical composition for preventing or treating an inflammatory disease, including the above-described anti-inflammatory active composition.

As used herein, the term “anti-inflammatory activity” refers to the suppression of inflammation, and the inflammation, which is a defense response of biological tissues to a certain stimulus, refers to a complex lesion that involves three intercurrent factors: tissue degeneration, circulatory disturbances and exudation, and tissue proliferation. More specifically, inflammation is a part of innate immunity, and as in other animals, human innate immunity recognizes cell surface patterns specific to pathogens. Phagocytes recognize cells with such surfaces as non-self and attack the pathogens. When a pathogen breaks the physical barrier and invade the body, an inflammatory response occurs. The inflammatory response is a non-specific defense mechanism that creates a hostile environment for microorganisms that have invaded an wound site. In the inflammatory response, when the body is wounded or an external infectious agent enters the body, white blood cells responsible for the initial immune response swarm and express cytokines. Therefore, the level of cytokine expression within cells serves as an indicator of inflammatory response activation.

In one embodiment of the present invention, the anti-inflammatory activity of the present invention may be due to microvesicles, exosomes, or cell secretomes included in the composition of the present invention, and more specifically, may be due to exosomes, but is not limited thereto.

As used herein, the term “inflammatory disease” may be any one selected from the group consisting of dermatitis, allergy, atopy, asthma, conjunctivitis, periodontitis, rhinitis, otitis media, pharyngitis, tonsillitis, pneumonia, gastric ulcer, gastritis, Crohn's disease, colitis, gout, ankylosing spondylitis, rheumatic fever, lupus, fibromyalgia, psoriatic arthritis, osteoarthritis, rheumatoid arthritis, periarthritis of the shoulder joint, tendinitis, tenosynovitis, peritendinitis, myositis, hepatitis, cystitis, nephritis, Sjogren's syndrome, multiple sclerosis, and acute or chronic inflammatory diseases, but is not limited thereto.

As used herein, the term “prevention” refers to any act of suppressing symptoms or delaying the onset of an inflammatory disease by administering a composition, and “treatment” or “amelioration” refers to any act of ameliorating or beneficially changing symptoms of an inflammatory disease by administering a composition.

Furthermore, as used herein, the term “quasi-drug” refers an article corresponding to one of: fibers, rubber products, or similar products used for the purpose of treatment, alleviation, care, or prevention of human or animal diseases; non-appliance, non-machinery, or similar articles that have insignificant influences on or do not directly act upon human bodies; and medication for sterilization, insecticide, and other similar uses for the purpose of preventing infectious diseases, excluding articles, other than appliances, machinery, or equipment, used for the purposes of diagnosis, treatment, alleviation, care, or prevention of diseases of human beings or animals; and articles, other than appliances, machinery, or equipment, used for the purpose of exerting pharmacological effects upon the structure or functions of human beings or animals.

Meanwhile, the term “excipient” usually refers to a substance added to a drug to impart an appropriate hardness or shape, or to impart a certain weight in the case where the amount of the main ingredient is small, in order to make it a size that is easy to handle. However, in the present invention, an “excipient (also called an additive)” known in the art provides the function of efficiently separating EVs from impurities such as cell debris, waste, proteins, and large particles. As non-limiting examples of the present invention, an excipient may be at least one selected from the group consisting of sucrose, trehalose, lactose, lactulose, maltose, cellobiose, isomaltose, turanose, dextrose, glucose, galactose, fructose, talose, mannose, tagatose, psicose, erythrose, erythrulose, threose, erythritol, arabinose, xylose, lyxose, ribose, ribulose, xylulose, aldoheptose, heptulose, octulose, gentianose, umbelliferose, planteos, isolychnose, raffinose, lychnose, stachyose, verbascose, mannitol, maltitol, lactitol, maltotriose, polyethylene glycol, xylitol, sorbitol, starch hydrolyzate, starch hydrolyzate reducing alcohol, nonionic surfactants (e.g., Tween-20, Triton X-100, Pluonic F-68, etc.), histidine, glycerol, and cholesterol.

In the composition of one embodiment of the present invention, the type of stem cell is not limited, but it may preferably be tonsil-derived stem cells but is not limited thereto. The type of stem cell is not limited as long as it does not pose a risk of infection by pathogens and does not cause an immune rejection response, but may preferably be tonsil-derived stem cells.

The composition of one embodiment of the present invention can be effectively used to prevent, suppress, alleviate, or ameliorate an inflammatory disease.

The pharmaceutical composition of the present invention may be prepared in a unit dose form or may be prepared by placing the composition in a multi-dose container by formulating the composition using a pharmaceutically acceptable carrier and/or excipient according to a method that may be easily implemented by one of ordinary skill in the art to which the present invention pertains.

Pharmaceutically acceptable carriers included in the pharmaceutical composition of the present invention are those commonly used in formulation, and may include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, and mineral oil, but are not limited thereto.

In addition to the above-mentioned ingredients, the pharmaceutical composition of the present invention may further include lubricants, wetting agents, sweeteners, flavoring agents, emulsifiers, suspending agents, preservatives, and the like. Suitable pharmaceutically acceptable carriers and formulations are described in detail in Remington's Pharmaceutical Sciences (19th ed., 1995).

The pharmaceutical composition of the present invention may be administered orally or parenterally. In the case of parenteral administration, the pharmaceutical composition may be administered by intravenous injection, subcutaneous injection, intramuscular injection, intraperitoneal injection, intradermal administration, topical administration, intranasal administration, intrapulmonary administration, and rectal administration. When administered orally, since proteins or peptides are digested, the oral composition must be formulated to coat the active agent or protect it from degradation in the stomach. In addition, the pharmaceutical composition may be administered by any device that allows the active agent to travel to a target cell.

The appropriate dosage of the pharmaceutical composition of the present invention varies depending on factors such as the formulation method, administration method, patient's age, weight, sex, pathological condition, food, administration time, administration route, excretion rate, and response sensitivity, and a generally skilled physician may easily determine and prescribe a dosage effective for the desired treatment or prevention. According to a preferred embodiment of the present invention, the daily dosage of the pharmaceutical composition of the present invention is 0.001 to 100 mg/kg. As used herein, the term “pharmaceutically effective amount” refers to an amount sufficient to prevent or treat an inflammatory disease.

The pharmaceutical composition of the present invention may be administered as an individual preventive or therapeutic agent, or in combination with other therapeutic agents, and may be administered sequentially or simultaneously with conventional therapeutic agents.

The concentration and/or content of the pharmaceutical composition of the present invention is not particularly limited, as long as it includes the above-described anti-inflammatory active composition.

In addition, the present invention provides an anti-inflammatory functional cosmetic composition including the above-described anti-inflammatory active composition.

The cosmetic composition is not particularly limited in any particular form, as long as it is suitable for use in the prevention or amelioration of an inflammatory disease. For example, the cosmetic composition may be formulated as a solution, suspension, emulsion, paste, gel, cream, lotion, powder, soap, surfactant-containing cleansing agent, oil, powder foundation, emulsion foundation, wax foundation, and spray, but is not limited thereto. More specifically, the cosmetic composition may be formulated as a softening toner, nourishing toner, nourishing cream, massage cream, essence, eye cream, cleansing cream, cleansing foam, cleansing water, pack, spray, or powder.

When the formulation of the present invention is a paste, cream, or gel, and animal oil, a vegetable oil, wax, paraffin, starch, tragacanth, cellulose derivatives, polyethylene glycol, silicone, bentonite, silica, talc, zinc oxide, or the like may be used as a carrier ingredient. When the formulation is powder or a spray, lactose, talc, silica, aluminum hydroxide, calcium silicate, or polyamide powder may be used as a carrier ingredient, and in particular, when the formulation is a spray, it may further include a propellant such as chlorofluorohydrocarbon, propane/butane, or dimethyl ether, but it is not limited thereto. When the formulation of the present invention is a solution or emulsion, a solvent, a solubilizer or an emulsifier is used as a carrier ingredient, and examples include water, ethanol, isopropanol, ethyl carbonate, ethyl acetate, benzyl alcohol, benzyl benzoate, propylene glycol, 1,3-butyl glycol oil, glycerol aliphatic esters, polyethylene glycol, and fatty acid ester of sorbitan. When the formulation is a suspension, a liquid diluent such as water, ethanol, and propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, or tragacanth may be used as a carrier ingredient. When the formulation of the present invention is a surfactant-containing cleansing agent, aliphatic alcohol sulfate, aliphatic alcohol ether sulfate, sulfosuccinic acid monoester, isethionate, imidazolinium derivatives, methyl taurate, sarcosinate, fatty acid amide ether sulfate, alkylamido betaine, aliphatic alcohols, fatty acid glyceride, fatty acid diethanolamide, vegetable oils, lanolin derivatives, or ethoxylated glycerol fatty acid ester may be used as a carrier ingredient, but it is not limited thereto. The ingredients included in the cosmetic composition of the present invention may include, in addition to the active ingredient and the carrier ingredient, ingredients commonly used in cosmetic compositions, and may include, for example, conventional auxiliary agents such as antioxidants, stabilizers, solubilizers, vitamins, pigments, and fragrances.

In addition, the present invention provides a health functional food composition for preventing or ameliorating an inflammatory disease, including the above-described anti-inflammatory active composition.

The types of the health functional food are not particularly limited as long as they are commonly manufactured and/or sold. For example, health functional food includes meat, sausage, bread, chocolate, candy, snacks, confectionery, pizza, ramen, other noodles, gum, dairy products including ice cream, various soups, beverages, tea, drinks, alcoholic beverages, and vitamin complexes, and may be used in the form of pills, powder, granules, infusions, tablets, capsules, or beverages, and include all health functional foods in the conventional sense.

The health beverage composition of the present invention has no particular limitations on the liquid component other than containing the anti-inflammatory active composition, and may contain various flavoring agents or natural carbohydrates as additional components, like a conventional beverage.

Typically, the amount of the anti-inflammatory active composition included in the health functional food composition may be 0.1% to 50% by weight, preferably 1% to 40% by weight, based on the total food weight. In addition, for long-term consumption for health and hygiene purposes or health control purposes, the amount may be below the above-described range, and since there are no safety issues, the active ingredient may also be used in an amount exceeding the above-described range.

In addition, the present invention also provides an quasi-drug composition for preventing or ameliorating an inflammatory disease, including the above-described anti-inflammatory active composition.

The anti-inflammatory active composition of the present invention may be added to an quasi-drug composition for the purpose of preventing or ameliorating an inflammatory disease. When the anti-inflammatory active composition of the present invention is used as an quasi-drug additive, the anti-inflammatory active composition may be added directly or combined with other quasi-drugs or quasi-drug ingredients, and may be used appropriately according to conventional methods. The amount of the active ingredient may be appropriately determined depending on the intended use (prevention, health, or therapeutic treatment). The quasi-drug composition is not particularly limited as long as it is typically used for the prevention or amelioration of an inflammatory disease. For example, the quasi-drug composition may be used in the manufacture of disinfectants, shower foams, mouthwashes, wet wipes, detergent soaps, hand washes, humidifier fillers, masks, ointments, coatings, or filter fillers.

The present invention is described in more detail in the following examples. However, the following examples are intended to illustrate the present invention and not to limit or restrict its scope. Anything readily inferred by those skilled in the art from the detailed description and examples of the present invention is deemed to fall within the scope of the present invention. References cited herein are hereby incorporated by reference.

### Example 1. Cell Culture

First, tonsil tissue (tonsil) was placed in a 50-ml tube containing 15 to 30 ml of 70% ethanol for one minute. Thereafter, the ethanol was removed, and the tissue was washed twice with 30 ml of 1× phosphate-buffered saline (PBS) to remove residual ethanol. After removing the PBS, the tonsil tissue was then transferred to a petri dish, and 5 ml of PBS was poured over the tissue.

The tonsil tissue was crushed using tweezers, the solution containing T cells and B cells was removed, and 5 ml of fresh PBS was added. The tonsil tissue was further crushed using tweezers to release a significant portion of lymphocytes from the tissue. After most of the solution was removed from the petri dish, when only the connective tissue remained, the tissue was cut into small pieces less than 5 mm in size with surgical scissors. The cut tissue was transferred to a 50 ml tube, and collagenase and DNase I were added to a total volume of 10 to 20 ml. The resulting mixture was incubated in a shaking incubator at 37 °C for 30 to 60 minutes. Thereafter, a cell strainer was attached to a new 50 ml tube, and the cell suspension was slowly transferred from the 50 ml tube containing the tissue, and then additional PBS (20 ml) was added on top of the tissue remaining in the strainer to recover the remaining cells.

The cells recovered as described above were centrifuged at 300×g for five minutes to remove the supernatant, the cell pellet was suspended in 5 ml of culture medium, and a Percoll concentration gradient was used to recover the cells.

First, the cells were carefully suspended on the upper layer of 15% Percoll medium and centrifuged at 500 to 900×g for 10 to 30 minutes, and then low-density cells located between 15% and 30% were collected and placed in a new tube and washed once with PBS. Thereafter, the cells were centrifuged at 300×g for five minutes, and the cell pellet was added to the culture medium to create a cell suspension. The cells were counted with a hemocytomer and suspended at a concentration of 10⁶/ml. Thereafter, 15 ml of the suspension was cultured in a 75 cm² flask or 10 ml in a 100 mm dish. At this time, the culture medium was replaced with fresh medium every three days, and subculture was performed when the fibroblast lineage cells reached a confluency of 70% or more. The subculture was performed at a number of 1,000 to 5,000/cm² depending on the surface area of the culture vessel.

### Example 2. Isolation of EVs From Tonsil-Derived Mesenchymal Stem Cell Culture

The tonsil-derived mesenchymal stem cells obtained in Example 1 were cultured in a culture medium containing fetal bovine serum. When the culture vessel reached a confluency of 80% or more, the existing medium was removed, cells were washed at least three times with PBS, and replacement culture with serum-free medium was initiated.

At this time, polydeoxyribonucleotide (PDRN) was added at a concentration of 1 to 100 μg/ml to stimulate tonsil-derived mesenchymal stem cells for 1 to 24 hours. The culture medium was then removed, PDRN was washed out, and the cells were cultured in serum-free medium or continuously cultured without replacement of fresh medium. Typically, after 24 hours of culture, the cells were washed, the medium was replaced, and then the cells were cultured for an additional 48 hours to carry out an exosome extraction step.

PDRN is known to have anti-inflammatory effects, aid wound healing and vascular regeneration, and possess moisturizing and anti-aging effects. Accordingly, tonsil-derived mesenchymal stem cells stimulated with PDRN may exhibit enhanced anti-inflammatory efficacy.

After culture was completed and all culture medium was recovered, cells and cell debris were removed by centrifugation at 300 to 900×g for 5 to 10 minutes.

Thereafter, the supernatant (conditioned medium, CM) was concentrated 10 to 50 times using a Minimate tangential flow filtration (TFF, Pall Corporation) system and centrifuged at 10,000-20,000×g for one to two hours at 4 °C using a high-speed centrifuge (Himac CR 22N, Himac-Science, Rotor Number P32ST, Takeda, Hitachinaka city, Japan).

After centrifugation, the supernatant was transferred to a separate tube. PBS or saline was added to the pellet fraction, which mainly consisted of microvesicles, in an amount equal to the volume of the pre-centrifuged concentrate. The pellet fraction was then suspended using a pipette and stored frozen until further experiments.

The separately recovered supernatant was centrifuged at 100,000 to 200,000×g for one to two hours at 4 °C using an ultracentrifuge (Himac CP 80NX, Himac-Science, Rotor Number P32ST, Takeda, Hitachinaka city, Japan).

After ultracentrifugation, the supernatant (cell secretion fraction) was recovered. The pellet, primarily composed of exosomes, was resuspended by adding PBS or saline in an amount equal to the pre-centrifugation concentrate. Each fraction was stored in the freezer until use in the experiment.

### Example 3. Characterization of EV Fraction From Tonsil-Derived Mesenchymal Stem Cells

As in Example 2, microvesicle fraction, exosome fraction, and secretome fraction isolated from the culture medium of tonsil-derived mesenchymal stem cells were analyzed by nanoparticle tracking analysis (NTA) using a NanoSight model NS300 (Malvern Instruments LTD., Malvern, UK) equipped with a red laser and a Scientific Complementary Metal-Oxide-Semiconductor (SCMOS) camera (Malvern Instruments LTD., Malvern, UK). The samples were diluted in 1 ml of PBS and mixed well, and then the diluted samples were injected into the laser chamber. Data analysis was performed with NTA v3.2.

As a result, as shown in FIG. 1A, it was confirmed that the conditioned medium was a mixture of particles of various sizes with the number of particles per milliliter being 1×10⁹, a step prior to high-speed centrifugation and ultracentrifugation

Meanwhile, as shown in FIG. 1B, the microvesicle fraction contained an average of 1.2×10⁸ particles per milliliter with an average particle size of 150 nm, confirming a mixture of particles of various sizes.

Furthermore, as shown in FIG. 2A, the exosome fraction contained an average of 2.4×10⁸ particles per milliliter with particles having an average particle size of 100 nm, as the main component.

In addition, as shown in FIG. 2B, the secretome fraction contained 7.2×10⁹ particles per milliliter with an average particle size of less than 50 nm, which was the typical size of exosomes.

### Example 4. Confirmation of Anti-Inflammatory Effect

### 4-1. RNA Extraction and Real-Time PCR (qPCR)

To confirm the anti-inflammatory effect of the present invention, human peripheral blood was collected from healthy donors. The blood sample was mixed with PBS at a 1:1 ratio. The resulting mixture was then overlaid on the upper layer of 20 ml of Ficoll Hypaque (Cytiva) solution (specific gravity 1.077 g/ml) in a 50 ml tube. The mixture was centrifuged at 900×g for 20 minutes, and the middle layer was recovered to extract peripheral blood mononuclear cells (PBMCs).

After collecting the PBMC layer as described above, centrifugation was repeated twice at 300×g for 5 to 10 minutes, and the pellet cells were suspended in RPMI-1640 (Cytiva HyClone RPMI, Logan, Utah, U.S.) supplemented with 10% fetal bovine serum (FBS) and 1% penicillin/streptomycin and used in the experiment.

The recovered PBMCs were prepared at a concentration of 3×10⁶/ml and dispensed 1 ml into each well of a 6-well plate. 2 ml of medium was added to the negative control group, and 1 ml of medium containing lipopolysaccharide (LPS) (300 ng/ml) was administered to all wells except the negative control group. Thereafter, 1 ml of tonsil-derived mesenchymal stem cells culture concentrate (final 5% to 20%), microvesicle concentrate (final 5% to 20%), exosome concentrate (final 5% to 20%), or cell secretion fraction (final 5% to 20%) was immediately added to the LPS-treated wells.

The LPS-stimulated PMBCs were incubated in a CO₂, 37 °C incubator for 3 to 12 hours. Thereafter, the attached PMBCs were collected from the bottom using a cell scraper or trypsin/EDTA solution. The cells were collected by centrifugation at 300×g for five minutes, and RNA was isolated for RT-qPCR.

4-2. Confirmation of TNF-α Production Inhibiting Effect To analyze the anti-inflammatory effects of the anti-inflammatory composition including as an active ingredient EVs isolated from tonsil-derived mesenchymal stem cells of the present invention, the target genes were set to TNF-α and IL-10, which are known inflammatory proteins, and the following experiments were conducted to observe changes in their expression.

First, RNA was extracted from cells recovered as in Example 4-1 above using the BIONEER AccuPrep Universal RNA Extraction kit, and then 1 μg of RNA per condition was treated with 10 pmol of oligo-dT primer and dNTP mixture (each 10 mM) at 65 °C for five minutes. Thereafter, cDNA was synthesized at 42 °C for 30 to 60 minutes in a total volume of 20 μl using the cDNA Synthesis Kit (Takara) by adding 1× buffer, 200 UM-RTase, and 20 U RNase inhibitor. Then, the synthesized cDNA was used to perform real-time PCR using FastFire qPCR PreMix (SYBR Green, TIANGEN, Beijing, China) on a QuantStudio 3 Real-Time PCR Instrument (Thermo Fisher) under the following conditions: Hold Stage: 95 °C, 1 min, PCR Stage: 40 cycles (step 1:95 °C, 5 sec; step 2:60 °C 15 sec), Melt Curve Stage: step 1:95 °C, 15 sec; step 2:60 °C, 1 min; step 3 (dissociation): 95 °C, 15 sec.

The expression level of TNF-α was averaged by glyceraldehyde 3-phosphate dehydrogenase (GAPDH), and the ΔCt and ΔΔCₜ values were obtained, and the displayed relative quantification (RQ) values were expressed using the 2^{-ΔΔct} calculation method. At this time, the primers used were GAPDH (FW: 5′GGAGCGAGATCCCTCCAAAAT-3′, RV: GGCTGTTGTCATACTTCTCATGG-3′) and 5-TNF-α (FW: 5′-CCCCCAGGGACCTCTCTCTAATC-3′, RV: ‘GGRTTGCTACAACATGGGCTACA-3’).

Thereafter, LPS-stimulated PBMCs were treated with each fraction of the culture medium from tonsil-derived mesenchymal stem cells, and their anti-inflammatory effects were analyzed by real-time PCR.

As shown in FIG. 3, the anti-inflammatory composition including as an active ingredient EVs isolated from tonsil-derived mesenchymal stem cells of the present invention was confirmed to effectively reduce LPS-induced TNF-α. Specifically, as shown in FIG. 3, RT-PCR revealed that the TNF-α level in the conditioned medium (CM), microvesicle, and exosome-treated experimental groups was all lower than that of the control group treated only with LPS in this order, with exosomes, microvesicles, and CM showing the lowest TNF-α level in this order. In particular, it was confirmed that the experimental group treated together with exosomes exhibited a superior inhibitory effect on TNF-α production.

### 4-3. Confirmation of IL-10 increasing effect

To confirm the effect of inducing the expression of IL-10, an anti-inflammatory cytokine, the following experiment was conducted.

First, PBMCs were treated with LPS and culture medium fractions of tonsil-derived mesenchymal stem cells. To quantify IL-10 secreted by the stimulated PBMCs and contained in the culture medium, 5×10⁵ cells/well were plated in a 24-well plate, treated with 100 ng/ml of LPS and 1× of the culture medium of the isolated tonsil-derived mesenchymal stem cells, cultured for 24 hours in a 37 °C incubator (5% CO₂). After 24 hours, the culture medium was recovered and centrifuged at 300×g for five minutes at 4 °C to remove any remaining cells. The IL-10 Standard ABTS ELISA Development Kit (PEPROTECH, Rocky Hill, NJ, USA) was used for measurement.

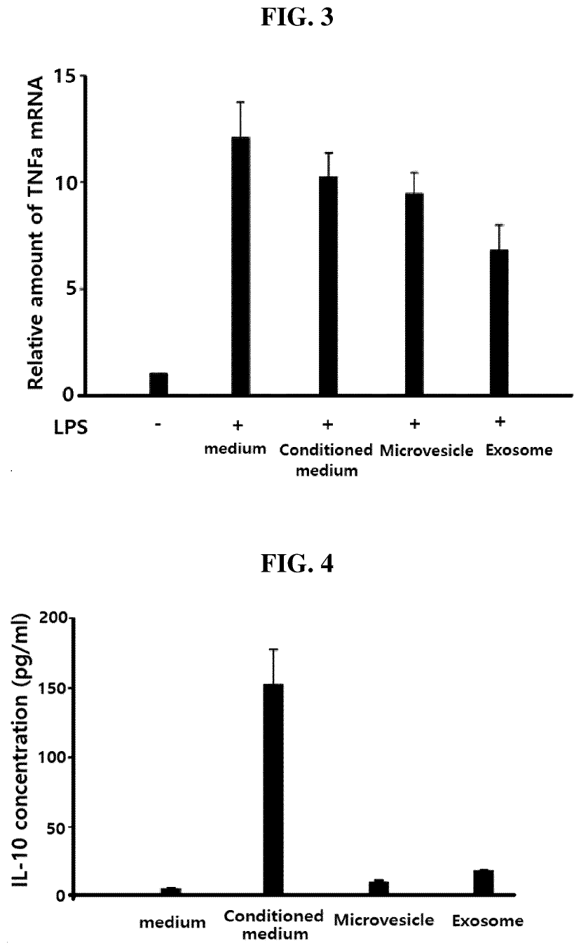

At this time, as a resulting of examining whether the residual IL-10 content in the culture medium itself affected the assay results, as shown in FIG. 4, IL-10 was almost absent in the microvesicle and exosome fractions, confirming that the increased IL-10 produced by exosomes and microvesicles under LPS-stimulated conditions was produced by the stimulated PBMCs.

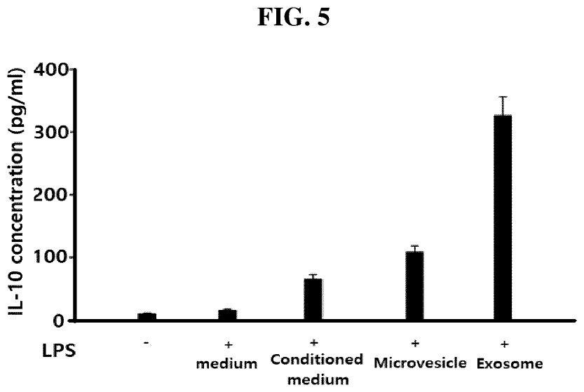

As shown in FIG. 5, the experimental results on the anti-inflammatory cytokine IL-10 expression inducing effect of the composition of the present invention indicated that IL-10, known as a key inhibitor of inflammation, exhibited the strongest expression inducing effect in the exosome fraction. In addition, it was revealed that IL-10 production was very low in the LPS-stimulated PBMCs, whereas expression was strongly induced in the microvesicle fraction and exosome fraction. In particular, it was confirmed that the expression effect was excellent in the exosome fraction, indicating that the exosome fraction exhibits the strongest inhibitory effect on skin inflammation.

### Example 5. Preparation of Freeze-Dried Powder Formulation Using Hyaluronic Acid (HA)

HA was added to exosomes isolated from the culture medium of the tonsil-derived mesenchymal stem cells, as described in Example 2, and the resulting mixture was freeze-dried to produce a freeze-dried exosome formulation.

Specifically, 3 μl of 1 million dalton HA was added to 6 μl of an exosome solution obtained by thawing frozen exosomes. Sterile distilled water was added, and the resulting mixture was stirred. 3 mL of an exosome+hyaluronic acid solution (exosomes: 0.2%, hyaluronic acid: 0.1%, sterile distilled water: 99.7%) was filled into a glass vial.

A powder-type exosome freeze-dried formulation was prepared by freeze-drying the exosome+hyaluronic acid solution in a glass vial at −45 °C.

In addition, the freeze-dried glass vial was sealed with a rubber seal for vacuum packing, thereby producing a freeze-dried and vacuum-packed exosome powder vial product.

The individual features of the various embodiments of the present invention may be partially or wholly coupled or combined with each other, and as can be fully understood by those skilled in the art, various technical connections and operations are possible, and each embodiment may be implemented independently of each other or may be implemented together in an associated relationship.

Although the embodiments of the present invention have been described in more detail with reference to the attached drawings, the present invention is not necessarily limited to these embodiments, and various modifications may be implemented without departing from the technical spirit of the present invention. Therefore, the embodiments disclosed in the present invention are not intended to limit the technical spirit of the present invention, but to explain, and the scope of the technical spirit of the present invention is not limited by these embodiments. Therefore, it should be understood that the above-described embodiments are exemplary in all aspects and not restrictive. The protection scope of the present invention should be interpreted by the claims below, and all technical ideas within a scope equivalent thereto should be interpreted as being included in the scope of the rights of the present invention.

1. An anti-inflammatory active composition comprising as an active ingredient extracellular vesicles isolated from tonsil-derived mesenchymal stem cells stimulated with polydeoxyribonucleotide (PDRN).

2. The anti-inflammatory active composition of claim 1, wherein the extracellular vesicles have an average diameter of 10 to 400 nm.

3. The anti-inflammatory active composition of claim 1, wherein the anti-inflammatory active composition has inflammatory cytokine TNF-α inhibiting activity.

4. The anti-inflammatory active composition of claim 1, wherein the extracellular vesicles have anti-inflammatory cytokine IL-10 increasing activity.

5. A pharmaceutical composition for preventing or treating an inflammatory disease, comprising the anti-inflammatory active composition of claim 1.

6. The pharmaceutical composition for preventing or treating an inflammatory disease of claim 5, wherein the inflammatory disease is any one selected from the group consisting of dermatitis, allergy, atopy, asthma, conjunctivitis, periodontitis, rhinitis, otitis media, pharyngitis, tonsillitis, pneumonia, gastric ulcer, gastritis, Crohn's disease, colitis, gout, ankylosing spondylitis, rheumatic fever, lupus, fibromyalgia, psoriatic arthritis, osteoarthritis, rheumatoid arthritis, periarthritis of the shoulder joint, tendinitis, tenosynovitis, peritendinitis, myositis, hepatitis, cystitis, nephritis, Sjogren's syndrome, multiple sclerosis, and acute or chronic inflammatory diseases.

7. An anti-inflammatory functional cosmetic composition comprising the anti-inflammatory active composition of claim 1.

8. A health functional food composition for preventing or ameliorating an inflammatory disease, comprising the anti-inflammatory active composition of claim 1.

9. A quasi-drug composition for preventing or ameliorating an inflammatory disease, comprising the anti-inflammatory active composition of claim 1.
